(19) United States
(12) Patent Application Publication
(10) Pub. No.: US 2009/0100544 A1
(43) Pub. Date: Apr. 16, 2009

(54) Drought Tolerant Corn with Reduced Mycotoxin

(76) Inventors: Donald Anstrom, Pawcatuck, CT (US); Bruce Hammond, Charles, MO (US); John Headrick, Newbury Park, CA (US); Jacqueline E. Heard, Webster Grove, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 12/248,950

(22) Filed: Oct. 10, 2008

Related U.S. Application Data
(60) Provisional application No. 61/124,803, filed on Oct. 11, 2007.

(51) Int. Cl. A01H 5/10; C12N 15/82; C12N 15/29; C12N 5/10; A01H 5/00

(52) U.S. Cl. 800/279; 536/23.6; 435/419; 800/301

(57) ABSTRACT

Transgenic corn plants having recombinant DNA for expressing a protein or proteins that provides water-deficit tolerance have improved yield under water deficit conditions and improved fungal resistance, and exhibit lower levels of colonization by mycotoxins in grain that is harvested from plants that experience water deficit tolerance.

### CROSS-REFERENCE TO RELATED APPLICATIONS

This application claims the benefit of priority of prior filed U.S. Application Ser. No. 61/124,803 filed Oct. 11, 2007, which is herein incorporated by reference in its entirety.

### FIELD OF THE INVENTION

Disclosed herein are transgenic plants that offer resistance to fungal infection and increased yield under water deficit stress and methods of making and using such plants.

### BACKGROUND OF THE INVENTION

There is a need to provide corn plants with enhanced yield, drought tolerance and resistance to mycotoxins.

### SUMMARY OF THE INVENTION

This invention provides fungal resistant transgenic crop plants where fungal resistance is imparted by recombinant DNA expressing one or more proteins that provide water-deficit tolerance or heat tolerance. Such proteins are selected from the group consisting of a cold shock protein, a cold binding factor, a NF-YB transcription factor (Hap3 CAAT box DNA binding transcription factor), or a combination thereof. One aspect of the invention provides aflotoxin-resistant corn seed. Another aspect of the invention provides a method of reducing fungal resistance in corn seed grown in environments containing air-born fungal spores of Aspergillus, Alternaria, Fusarium and Penicillium, by producing said corn seed from transgenic plants having recombinant DNA that expresses one or more proteins that provide water-deficit tolerance or heat tolerance.

The invention also provides non-natural corn DNA in a corn cell comprising constructs for expressing two or more proteins selected from the group consisting of a bacterial cold shock protein, a cold binding transcription factor and an NF-YB transcription factor. In one embodiment, the non-natural corn DNA comprises recombinant DNA for expressing a Bacillus subtilis cspB protein and recombinant DNA for expressing a corn NF-YB transcription factor protein.

### DETAILED DESCRIPTION OF PREFERRED EMBODIMENTS

As used herein “water deficit” means a period when water available to a plant is not replenished at the rate at which it is consumed by the plant. A long period of water deficit is colloquially called drought. Lack of rain or irrigation may not produce immediate water stress if there is an available reservoir of ground water for the growth rate of plants. Plants grown in soil with ample groundwater can survive days without rain or irrigation without adverse affects on yield. Plants grown in dry soil are likely to suffer adverse affects with minimal periods of water deficit. Severe water stress can cause wilt and plant death; moderate drought can cause reduced yield, stunted growth or retarded development. Plants can recover from some periods of water stress without significantly affecting yield. However, water stress at the time of pollination can have an irreversible effect in lowering yield. Thus, a useful period in the life cycle of corn for observing water stress tolerance is the late vegetative stage of growth before tasseling. Water stress tolerance requires comparison to control plants. For instance, plants of this invention can survive water deficit with a higher yield than control plants. In the laboratory and in field trials drought can be simulated by giving plants of this invention and control plants less water than an optimally-watered control plant and measuring differences in traits.

A suitable control plant may be a non-transgenic plant of the parental line used to generate a transgenic plant herein. A control plant may in some cases be a transgenic plant line that includes an empty vector or marker gene, but does not contain the recombinant polynucleotide of the present invention that is expressed in the transgenic plant being evaluated. A control plant in other cases is a transgenic plant expressing the gene with a constitutive promoter. In general, a control plant is a plant of the same line or variety as the transgenic plant being tested, lacking the specific trait-conferring, recombinant DNA that characterizes the transgenic plant. Such a progenitor plant that lacks that specific trait-conferring recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may include other recombinant DNA.

A transgenic “plant cell” means a plant cell that is transformed with stably-integrated, non-natural, recombinant DNA, e.g. by Agrobacterium-mediated transformation or by bombardment using microparticles coated with recombinant DNA. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

A “transgenic” plant or seed means one whose genome has been altered by the incorporation of recombinant DNA, e.g. by transformation, regeneration from a transformed plant or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant. A variety of plants can be advantageously transformed with recombinant DNA for expressing a protein to provide water stress tolerance and/or enhanced yield. Especially useful transgenic plants with water stress tolerance include corn (maize), soybean, cotton, canola (rape), wheat, rice, alfalfa, sorghum, grasses, vegetables and fruits.

“Expressing a protein” refers to the process by which cells transcribe recombinant DNA to mRNA and translate the mRNA to a protein. The recombinant DNA usually includes 5′ regulatory elements such as promoters and enhancer introns, as well as 3′ polyadenylation sites, introns, transit peptide DNA, markers and other elements commonly used by those skilled in the art.

“Recombinant DNA” means a DNA molecule that is made by combination of two otherwise separated segments of DNA, e.g., by chemical synthesis or by the manipulation of isolated segments of nucleic acids by genetic engineering techniques. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein that provides water deficit tolerance or heat tolerance (e.g. a cold shock protein, a cold binding factor protein, or an NF-YB protein) linked to a 3′ DNA element for providing a polyadenylation site and signal. Useful recombinant DNA also includes expression cassettes for expressing one or more proteins conferring herbicide tolerance and/or insect resistance. A useful expression cassette for expressing a cold shock protein comprises a rice tubulin A promoter linked to DNA encoding Bacillus subtilis cold shock protein B (B.subtilis cspB) and a rice tubulin A 3′ polyadenylation element. A useful expression cassette for expressing a NF-YB protein comprises a rice actin promoter linked to DNA encoding Zea mays NF-YB protein and an Agrobacterium transcript 7 3′ polyadenylation element. A useful expression cassette for expressing a glyphosate herbicide selectable marker comprises a rice actin promoter linked to DNA encoding a glyphosate resistant EPSPS protein and an Agrobacterium transcript nos 3′ polyadenylation element. Rice tubulin A promoter and 3′ elements are disclosed in U.S. Patent Application Publication 2005/0048566 A1; rice actin promoters are disclosed in U.S. Pat. No. 5,641,876; and Agrobacterium 3′ polyadenylation elements are disclosed in U.S. Pat. No. 6,090,627.

Plant pathogens include fungi, e. g. the fungi that cause powdery mildew, rust, leaf spot and blight, damping-off, root rot, crown rot, cotton boll rot, stem canker, twig canker, vascular wilt, smut, or mold, including, but not limited to, Fusarium spp., Phakospora spp., Rhizoctonia spp., Aspergillus spp., Gibberella spp., Pyricularia spp., Alternaria spp., and Phytophthora spp. More specific examples of fungal plant pathogens include Phakospora pachirhizi (Asian soy rust), Puccinia sorghi (corn common rust), Puccinia polysora (corn Southern rust), Fusarium oxysporum and other Fusarium spp., Alternaria spp., Penicillium spp., Pythium aphanidermatum and other Pythium spp., Rhizoctonia solani, Aspergillus flavus (Aspergillus ear rot), Exserohilum turcicum (Northern corn leaf blight), Bipolaris maydis (Southern corn leaf blight), Ustilago maydis (corn smut), Fusarium graminearum (Gibberella zeae), Fusarium verticilliodes (Gibberella moniliformis), F. proliferatur (G. fujikuroi var. intermedia), F. subglutinans (G. subglutinans), Diplodia maydis, Sporisorium holci-sorghi, Colletotrichum graminicola, Setosphaeria turcica, Aureobasidium zeae, Phytophthora infestans, Phytophthora soiae, Sclerotinia sclerotiorum.

Human and other animal foodstuffs are a major potential source of nutrients for fungi. Spores of a wide range of fungi are common in the air and, if conditions are suitable, fungi can colonize the foodstuffs. Fungi take from their environment nutrients which are used for their growth and development. When the energy resource becomes depleted, the production of secondary metabolites increases, including a variety of compounds which cause toxicosis in humans and other herbivores. Such compounds called mycotoxins are dangerous when they are ingested accidentally with food. Common toxins include alkaloids, cyclopeptides, and coumarins. The compounds are active at extremely low concentrations and have a rapid effect. The toxins may cause death. In sublethal quantities, the toxins may also trigger cancer, and influence the physiology of the consumer. Many of the compounds are heat stable remaining active after cooking or treatment of foodstuff. The potential for damage is particularly important for human foods, and food for livestock held in intensive conditions.

Some common air-borne fungi that are known to produce extremely toxic compounds include Aspergillus, Alternaria, Fusarium and Penicillium. These fungi can grow on stored grains and animal feeds especially when humidity is high. They can also grow in living plants of cotton, peanuts and corn, where colonization of the host plant may take place prior to seed ripening. Stress from insect or environmental damage can facilitate fungal infection of living plants. See Cassel et al., “Aflatoxins—Hazards in Grain/Aflatoxicosis and Livestock”, South Dakota State University Cooperative Extension Service, FS 907 which reports that “Below—normal soil moisture (drought stress) has also been found to increase the number of Aspergillus spores in the air. Therefore, when drought stress occurs during pollination, the increased inoculum load (spores in the air) greatly increases the chances of infection. Furthermore, drought stress, nitrogen stress and other stresses that affect plant growth during pollination can increase the level of aflatoxins produced by Aspergillus fungi. Often, Aspergillus will grow in the unfilled portions of the ear.” See Xu et al., 2003, “Progress toward developing stress—tolerant tolerant and low-aflatoxins corn hybrids for the southern states” [abstract], 16^{th} Annual Aflatoxin Elimination Workshop Proceedings, p. 63, which reports “Drought and heat tolerant corn have less grain molds under drought stress.” See Anderson et al., “Managing Drought—Drought Advisory for Corn Production”, North Carolina Cooperative Extension Service, AG 519-13 which states “When the crop is subjected to drought, Aspergillus actually moves down corn silks to infect kernels and produce toxins. . . . Any action to prevent corn from undergoing drought stress will reduce concentrations in grain.” Infection of corn via silks is also discussed by Diener et al., “Epidemology of Aflatoxin Formation by Aspergillus flavus, Ann. Rev. Phytopathol. 187, 25:249-70.

DNA constructs comprising promoters and cold shock proteins useful for transformation into plant cells for providing water deficit tolerance are disclosed in published patent application US 2005/0097640 A1. DNA constructs comprising promoters and cold binding factors useful for transformation into plant cells for providing water deficit tolerance are disclosed in U.S. Pat. No. 5,892,009. DNA constructs comprising promoters and NF-YB transcription factors (also called Hap3 transcription factors) useful for transformation into plant cells for providing water deficit tolerance are disclosed in published patent application US 2005/0022266 A1. The published applications also disclose transformation methods for introducing the DNA constructs into plant cells, methods of regenerating plants from transformed cells and methods of introgressing recombinant DNA from a regenerated plant into other plant lines.

The plants of this invention can be further enhanced with stacked traits, e.g., a crop having an enhanced agronomic trait resulting from expression of DNA disclosed herein, in combination with herbicide and/or pest resistance traits. For example, genes of the current invention can be stacked with other traits of agronomic interest, such as a trait providing herbicide resistance, or insect resistance, such as using a gene from Bacillus thuringiensis to provide resistance against lepidopteran, coleopteran, homopteran, hemiopteran, and other insects. Herbicides for which resistance is useful in a plant include glyphosate herbicides, dicamba herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and glufosinate herbicides. Persons of ordinary skill in the art are enabled in providing stacked traits by reference to U.S. 2003/0106096A1 and 2002/0112260A1 and U.S. Pat. Nos. 5,034,322; 5,776,760; 6,107,549 and 6,376,754 and to insect/nematode/virus resistance by reference to U.S. Pat. Nos. 5,250,515; 5,880,275; 6,506,599; 5,986,175 and U.S. 2003/0150017 A1.

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. Nos. 5,015,580 (soybean); 5,550,318 (corn); 5,538,880 (corn); 5,914,451 (soybean); 6,160,208 (corn); 6,399,861 (corn) and 6,153,812 (wheat) and Agrobacterium-mediated transformation is described in U.S. Pat. Nos. 5,159,135 (cotton); 5,824,877 (soybean); 5,591,616 (corn); and 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plant lines for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line.

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV) and gentamycin (aac3 and aacC4) or resistance to herbicides such as glufosinate (bar or pat) and glyphosate (aroA or EPSPS). Examples of such selectable are illustrated in U.S. Pat. Nos. 5,550,318; 5,633,435; 5,780,708 and 6,118,047. Selectable markers which provide an ability to visually identify transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻²s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or inbred or hybrid progeny plants for the enhanced trait. For efficiency a selection method is designed to evaluate multiple transgenic plants (events) having the recombinant DNA, for example multiple plants from 2 to 20 or more transgenic events. Transgenic plants grown from transgenic seed provided herein demonstrate improved agronomic traits that contribute to increased yield or enhanced water deficit tolerance or both.

Not all transgenic events will be in transgenic plant cells that provide plants and seeds with an enhanced or desired trait depending on factors, such as location and integrity of the recombinant DNA, copy number, unintended insertion of other DNA, etc. As a result transgenic plant cells of this invention are identified by screening transformed progeny plants for enhanced water deficit stress tolerance and yield. For efficiency a screening program is designed to evaluate multiple transgenic plants preferably with a single copy of the recombinant DNA from 2 or more transgenic events.

The following examples illustrates embodiments of the invention.

### EXAMPLE 1

This example describes construction of plant expression vectors used for transforming plant cells useful in the various aspects of the invention. Transgenic corn with recombinant DNA expressing a bacterial cold shock protein, i.e. cspB, is prepared as disclosed in US 2005/0097640 A1 and identified as imparting water deficit tolerance. The transgenic corn line is used to produce an inbred transgenic corn line that is crossed to another inbred corn line to produce progeny hybrid corn seed having the recombinant DNA. The hybrid seed is used to produce corn plants with transgenic plant cells that are grown in a water-deficit environment and inoculated with spores of Aspergillus flavus. As compared to control corn plants the grain from the transgenic hybrid plants have lower measurable aflatoxin.

### EXAMPLE 2

This example illustrates the preparation of non-natural corn DNA in a corn cell comprising constructs for expressing two or more proteins selected from the group consisting of a bacterial cold shock protein, a cold binding transcription factor and an NF-YB transcription factor and transgenic corn cells comprising such non-natural corn recombinant DNA and transgenic corn seed comprising such cells having the non-natural corn recombinant DNA and methods of using such seed to reduce fungal colonization of corn seed on corn plants grown in environments containing air-born fungal spores of Aspergillus, Alternaria, Fusarium or Penicillium.,

Seeds from two distinct transgenic corn plants with different female and male germplasm backgrounds are planted in alternating rows in a field. In odd numbered rows are planted seeds from a first transgenic, inbred male germplasm corn plant having cells comprising stably-integrated, non-natural recombinant DNA expressing a bacterial cold shock protein from Bacillus subtillus, i.e., as disclosed in WO05033318. This application and in particular, the disclosed cold shock protein sequences provided therein are incorporated herein by reference. In even numbered rows are planted seeds from a second transgenic female germplasm corn plant having cells comprising stably-integrated, non-natural recombinant DNA expressing an NF-YB transcription factor, i.e. as disclosed in US20080104730. The plants are grown to maturity and tassels from corn plants in the rows grown from seed from the female germplasm transgenic corn plant are removed before pollination, allowing pollen from the corn plants in the rows grown from seed from the male germplasm transgenic corn plant to pollinate plants in all rows. After pollination the pollen producing plants are cut down allowing the remaining plants to produce hybrid seed containing cells having stably-integrated, non-natural recombinant DNA that expresses both the bacterial cold shock protein and the NF-YB transcription factor. The hybrid seed is grown to maturity, harvested and saved for replanting.

The saved, transgenic corn seed having cells with stably-integrated, non-natural recombinant DNA for expressing bacterial cold shock protein and an NF-YB transcription factor are planted in one field to grow a crop of corn plants that are tolerant to water deficit stress. A separate field is planted with non-trangenic hybrid corn seed prepared by crossing non-transgenic female germplasm corn plants with non-transgenic male germplasm corn plants, as a control. Both fields are subjected to water deficit stress during the growing season at the time of pollination and during grain fill. Both fields are subjected to air-born fungal spores from natural fungus including Aspergillus, Altenaria, Fusarium and Penicillium fungi during the period from grain fill to harvest. At harvest the corn from each field is analyzed for the presence of fungal colonization and the corn harvested from the transgenic plants has significantly less fungal colonization as well as significantly higher yield. After several months of segregated storage under similar conditions the corn harvested from the transgenic plants has significantly less fungal colonization.

### EXAMPLE 3

This example illustrates alternative preparation of non-natural corn DNA in a corn cell comprising constructs for expressing two or more proteins selected from the group consisting of a bacterial cold shock protein, a cold binding transcription factor and an NF-YB transcription factor and transgenic corn cells comprising such non-natural corn recombinant DNA and transgenic corn seed comprising such cells having the non-natural corn recombinant DNA and methods of using such seed to reduce fungal colonization of corn seed on corn plants grown in environments containing air-born fungal spores of Aspergillus, Alternaria, Fusarium or Penicillium.

A callus from a transformable corn variety is transformed by Agrobacterium-mediated transformation using a plasmid vector containing a transcription unit for a selectable marker, a transcription unit for expressing a bacterial cold shock protein from Bacillus subtillus and a transcription unit for expressing an NF-YB transcription factor, where the transcription factors have the elements described in the above paragraph [0011].

A transformed cell is cultivated in a medium to promote growth into a corn plant which is allowed to produce seeds having cells comprising stably-integrated, non-natural recombinant DNA for expressing a bacterial cold shock protein from Bacillus subtillus and a transcription unit for expressing an NF-YB transcription factor. The recombinant DNA is introgressed into an elite, inbred corn line to produce seed having cells comprising stably-integrated, non-natural recombinant DNA for expressing a bacterial cold shock protein from Bacillus subtillus and a transcription unit for expressing an NF-YB transcription factor.

Seeds from the transgenic corn plants and seed from a non transgenic corn plant are planted in alternating rows in a field. In odd numbered rows are planted seeds from the transgenic, inbred corn plant having cells comprising stably-integrated, non-natural recombinant DNA expressing a bacterial cold shock protein from Bacillus subtillus and an NF-YB transcription factor. Non-transgenic seeds are planted in the even numbered rows. The plants are grown to maturity and tassels from corn plants in the rows grown from seed from the transgenic plant are removed before pollination, allowing pollen from the non transgenic corn plants to pollinate plants in all rows. After pollination the pollen producing plants are cut down allowing the remaining plants to produce hybrid seed containing cells having stably-integrated, non-natural recombinant DNA that expresses both the bacterial cold shock protein and the NF-YB transcription factor. The hybrid seed is grown to maturity, harvested and saved for replanting.

The saved, transgenic hybrid corn seed having cells with stably-integrated, non-natural recombinant DNA for expressing bacterial cold shock protein and an NF-YB transcription factor are planted in one field to grow a crop of corn plants that are tolerant to water deficit stress. A separate field is planted with non-trangenic hybrid corn seed with the same genetic background as a control. Both fields are subjected to water deficit stress during the growing season at the time of pollination and during grain fill. Both fields are subjected to air-born fungal spores from natural fungus including Aspergillus, Altenaria, Fusarium and Penicillium fungi during the period from grain fill to harvest. At harvest the corn from each field is analyzed for the presence of fungal colonization and the corn harvested from the transgenic plants has significantly less fungal colonization as well as significantly higher yield. After several months of segregated storage under similar conditions the corn harvested from the transgenic plants has significantly less fungal colonization.

### EXAMPLE 4

This example illustrates the preparation of non-natural corn DNA in a corn cell as described in Example 2 where the proteins expressed include a Bacillus subtilis CspB protein and a corn NF-YB transcription factor.

Hybrid corn seed is produced by crossing homozygous inbred lines of different corn male and female germplasm backgrounds, each of which contains non-natural corn DNA for expression of either a bacterial cold shock protein or an NF-YB transcription factor protein. The same male and female germplasms is used in production of all of the transgenic and non-transgenic lines. Seeds from transgenic homozygous inbred corn plants in a male germplasm that comprise recombinant DNA expressing a cold shock protein are planted in alternating rows in a field. Seed from transgenic homozygous inbred corn plants in a female germplasm that comprise recombinant DNA expressing an NF-YB transcription factor protein is planted in the other rows. Thus, in odd numbered rows are planted seeds from a transgenic homozygous inbred male germplasm corn plant having cells comprising stably-integrated, non-natural recombinant DNA expressing a bacterial cold shock protein from Bacillus subtillus, i.e., as disclosed in WO05033318, and in even numbered rows are planted seeds from a transgenic homozygous inbred female germplasm corn plant having cells comprising stably-integrated, non-natural recombinant DNA expressing a corn NF-YB transcription factor at low levels, i.e. as disclosed in WO08002480.

The plants are grown to maturity and tassels from corn plants in the rows grown from seed from the NF-YB female germplasm transgenic corn plant are removed before pollination, allowing pollen from the corn plants in the rows grown from seed from the cspB male germplasm transgenic plant to pollinate plants in all rows. After pollination the pollen producing plants are cut down allowing the remaining plants to produce hybrid seed containing cells having stably-integrated, non-natural recombinant DNA that expresses both the bacterial cold shock protein and the NF-YB transcription factor. The hybrid seed is grown to maturity, harvested and saved for replanting.

The above steps are repeated for production of additional hybrid seed lots by crossing different low level NF-YB expressing transgenic homozygous corn events (in the same female germplasm as used above) with the same cspB expressing homozygous inbred male germplasm corn plant event described above.

The saved hybrid transgenic corn seed having cells with stably-integrated, non-natural recombinant DNA for expressing Bacillus subtilis cspB protein and corn NF-YB transcription factor are planted and tested for effects of water deficit stress. Control hybrid seed is planted in the same fields. Control seed 1 (Hybrid entries 2, 4, 6 and 8) is from hybrid plants prepared by crossing each of the transgenic homozygous inbred female germplasm corn plant events expressing corn NF-YB at low levels with non-transgenic male germplasm corn plants. Control seed 2 (Hybrid entry 9) is from hybrid plants prepared by crossing the transgenic homozygous inbred male germplasm corn plants expressing Bacillus subtilis cspB protein with non-transgenic female germplasm corn plants. Control seed 3 (Hybrid entry 10) is from a non-transgenic hybrid control prepared by crossing male and female non-transgenic corn germplasm plants. Thus, test and control plants thus have the same genetic background except for the presence of transgenes in the cspB and NF-YB plants and the cspB or NF-YB expressing transgenic controls.

The hybrid corn seed was planted in replicated yield trials (6 locations with 3 replicates in each location). Control and transgenic events were planted at the same plant density and replication. To provide water deficit stress conditions, water was withheld from the corn plants during the V8-R2 stages of development. During the water deficit episode, the plants were monitored for visual symptoms of drought stress severity. Plants were “pulsed” with small amounts of water to ameliorate the severity of stress once significant AM leaf rolling was observed. Once the crop reached the R2 developmental stage of development, watering was resumed to full recovery through the remaining growing season.

Once the corn crop reached physiological maturity, i.e. 10-25% grain moisture, plots were harvested. Resulting grain yield was normalized to 15.5% moisture and expressed in terms of bushels/acre (bu/acre) and is reported in Table 1.

TABLE 1

| | Recombinant | Recombinant | |
|---|---|---|---|
| Hybrid | DNA in Male | DNA in Female | Yield |
| Entry | Parent | Parent | (Bu/acre) |
| 1 | cspB | NF-YB Event 1 | 174.43* |
| 2 | None | NF-YB Event 1 | 165.93 |
| 3 | cspB | NF-YB Event 2 | 166.07 |
| 4 | None | NF-YB Event 2 | 167.95 |
| 5 | cspB | NF-YB Event 3 | 164.93* |
| 6 | None | NF-YB Event 3 | 152.62 |
| 7 | cspB | NF-YB Event 4 | 155.04 |
| 8 | None | NF-YB Event 4 | 153.05 |
| 9 | cspB | None | 163.55 |
| 10 | None | None | 152.68 |

*Events outperforming single gene transgenics and control

The above data demonstrate that hybrid transgenic corn seed comprising non-natural recombinant DNA for expression of a Bacillus subtilis cspB protein and for low level expression of a corn NF-YB transcription factor protein can be grown to produce a corn plant crop having greater yield increases under water deficit stress conditions than are obtained with corn seed comprising non-natural recombinant DNA for expression of either a Bacillus subtilis cspB protein or a corn NF-YB transcription factor protein alone. The harvested grain from the transgenic corn plants has significantly less fungal colonization than non-transgenic controls that are grown under water deficit stress.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations may be applied to the materials and methods described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of reducing fungal colonization of corn seed on corn plants grown in environments containing air-born fungal spores of Aspergillus, Alternaria, Fusarium or Penicillium, wherein said method comprises producing said corn seed from transgenic plants having recombinant DNA that expresses two or more proteins that provide water-deficit tolerance.

2. The method of claim 1 wherein said two or more proteins is selected from the group consisting of a cold shock protein, a cold binding factor, an NF-YB transcription factor, or a combination thereof.

3. The method of claim 2 for reducing fungal colonization of corn seed on corn plants grown in environments containing air-born fungal spores of Aspergillus, Alternaria, Fusarium or Penicillium, wherein said method comprises producing corn seed from water-deficit tolerant transgenic plants having cells with an altered genome containing stably-integrated, non-natural recombinant DNA that expresses a bacterial cold shock protein and an NF-YB transcription factor.

4. Non-natural corn DNA in a corn cell comprising constructs for expressing two or more proteins selected from the group consisting of a bacterial cold shock protein, a cold binding transcription factor and an NF-YB transcription factor.

5. The non-natural corn DNA in a corn cell of claim 4 wherein said constructs express a Bacillus subtilis cspB and a corn NF-YB transcription factor.

6. The non-natural corn DNA of claim 5 wherein said corn NF-YB transcription factor is expressed at low levels.

7. A transgenic corn cell comprising the non-natural corn recombinant DNA of claim 4.

8. A transgenic corn cell comprising the non-natural corn recombinant DNA of claim 5.

9. A transgenic corn seed comprising cells having the non-natural corn recombinant DNA of claim 4.

10. A transgenic corn seed comprising cells having the non-natural corn recombinant DNA of claim 5.

11. A crop of corn plants grown from the transgenic corn seed of claim 5, wherein said corn plants have improved yield under water deficit conditions and reduced fungal colonization as compared to control corn plants.
